(19) United States
(12) Patent Application Publication
(10) Pub. No.: US 2017/0119515 A1
(43) Pub. Date: May 4, 2017

(54) Surgical Implant

(71) Applicant: Ethicon, LLC, San Lorenzo, PR (US)

(72) Inventors: Jorg Priewe, Kiel (DE); Pierre Chantereau, Hamburg (DE); Kerstin Spychaj, Hamburg (DE)

(21) Appl. No.: 15/259,273

(22) Filed: Sep. 8, 2016

(30) Foreign Application Priority Data
Oct. 30, 2015 (DE) 10215013989.4

(51) Int. Cl. A61F 2/00

(52) U.S. Cl. CPC A61F 2/0063; A61F 2210/0004; A61F 2250/0085; A61F 2240/001

(57) ABSTRACT

A surgical implant (1) comprises a flexible, areal basic structure (2) having a first face (3) and a second face (4) and being provided with pores (6) extending from the first face (3) to the second face (4). An absorbable film layer (10) is placed at the first face (3) of the basic structure (2), is attached to the basic structure (2) and has an outer face (12) facing away from the basic structure (2). An absorbable marker (20) is attached to the outer face (12) of the film layer (10), wherein the marker (20) is adapted to indicate an upside/downside orientation of the outer face (12) of the film layer (10) and to indicate a center area of the basic structure (2).

The invention relates to a surgical implant, in particular to a tissue reinforcing implant, e.g. for repair of a tissue or muscle wall defect, such as a ventral hernia, and to a process of manufacturing such an implant.

The use of tissue reinforcing implants, such as polymer meshes, is widely spread. In 1995, a procedure was developed by F. Ugahary that combines the advantages of pre-peritoneal mesh fixation with the convenience of using small incisions for forming access openings.

For repairing inguinal hernias, laparoscopic techniques have been developed. One technique uses a trans-abdominal pre-peritoneal mesh-plasty (TAPP), wherein an implant mesh is pre-peritoneally positioned through a trans-abdominal laparoscopic access opening. Another technique is totally extra-peritoneal pre-peritoneal mesh-plasty (TEP), in which a large mesh is laparoscopically applied via an extra-peritoneal access opening. The implanted mesh covers all three potential hernia openings.

Often, after the intra-peritoneal implantation of polymer meshes, adhesions of internal structures occur, such as intestine, omentum, etc. Thus, there have been many efforts directed to providing implants that prevent, reduce the intensity of and/or minimize adhesions in the area of the implant, both in the center and in the periphery.

Tissue reinforcing implants, commonly referred to as areal implants, have been developed that match or compliment the mechanical properties of the underlying tissue. Orienting the areal implant relative to the underlying tissue may be important because both the target tissue and the implant may have mechanical properties that are anisotropic. One example of an implant having anisotropic properties is a mesh with reinforcing fibers running in only one direction.

A conventional implant may require the surgeon to place an alignment mark on, e.g., the mesh for aligning the mesh on the patient. Such alignment marks are made using a skin marker, which may easily wash off, however.

U.S. Pat. No. 7,615,065 B discloses an areal implant comprising a long-term stable, mesh-like basic structure having pores of a size in the range of from 1.5 mm to 8 mm, which is provided on both sides, at least in part, with a synthetic, absorbable polymer film. The two polymer films are glued or welded together in the pores of the basic structure. This implant reduces the formation of adhesions of internal structures in human or animal organisms and, after a period of time, facilitates tissue in-growth.

WO 2003/037215 A discloses an areal implant for reinforcing tissue comprising a mesh-like basic structure and an alignment marking in a central region that indicates the center of the implant. The central marking and a marking line running through the central marking are used for aligning the implant at a surgical opening.

U.S. Pat. No. 8,821,585 B describes a composite anisotropic tissue-reinforcing implant comprising an asymmetric marker between a film and a surgical mesh, which is aligned orthogonally to the mesh running direction.

US 2015/0057762 A discloses a surgical implant comprising markers having protrusions located behind an adhesion barrier film.

DE 295 12 361 U describes a surgical mesh without an adhesion barrier film, wherein the mesh sides have different colors. Such a mesh cannot be used intraperitoneally without causing adhesions.

WO 2013/126718 A discloses a surgical implant comprising a bioabsorbable incision reinforcement element, a long-term mesh, and a bioabsorbable coating disposed on the mesh. The reinforcement element can also include a marker.

U.S. Pat. No. 8,579,922 B describes a suture kit comprising a mesh having indicators which can be letters, words or symbols.

WO 2013/098348 A discloses a mesh patch having a central marker which can be coated with an adhesion barrier.

US 2014/0276999 A describes a mesh/film laminate, wherein the center area of the mesh is marked by a center marking and directional indicators point from the center marking to at least two corners of the outer periphery of the basic structure.

In spite of the advances achieved by the implants discussed so far, there remains a need for tissue reinforcing implants having anisotropic properties (e.g. with respect to elongation behavior) to simulate the anisotropic properties of the supported tissue (e.g. abdominal tissue). There also remains a need for tissue reinforcing implants that minimize or eliminate the occurrence of adhesions. In addition, there remains a need for tissue reinforcing implants having relatively durable alignment markers. There also remains a need for tissue reinforcing implants that may be used for intra-peritoneal or laparoscopic applications, that fit through a trocar sleeve, that are simple to deploy, that may be fixable with sutures, tacks, or glues, and that have an absorbable orientation marker allowing the right positioning and identifying the right implant side during operation.

The object of the invention is to provide a surgical implant, in particular for intra-peritoneal tissue reinforcement in laparoscopic or open surgery, which permits an easy and safe realization whether it is placed in the correct orientation.

This object is achieved by the surgical implant according to claim 1. Claim 17 relates to a process of manufacturing such a surgical implant. Advantageous versions of the invention follow from the dependent claims.

The surgical implant according to the invention comprises a flexible, areal basic structure having a first face and a second face (i.e., a first side and a second side). The term “areal” means that the basic structure is generally flat, i.e. that it has a relatively small thickness, but because the basic structure is flexible it can be deformed into the third dimension. The basic structure is provided with pores extending from the first face to the second face. An absorbable film layer is placed at the first face of the basic structure, is attached to the basic structure and has an outer face facing away from the basic structure. An absorbable marker is attacked to the outer face of the film layer. The marker is adapted to indicate an upside/downside orientation of the outer face of the film layer and to indicate a center area of the basic structure. “Upside/downside orientation” relates to the orientation whether the outer face of the film layer is at the top side or at the bottom side when the implant is placed on a desk.

The absorbable film layer may be designed as a barrier layer which generally has an anti-adhesive effect and prevents bodily tissue from growing into the basic structure via the first face thereof. Since the film layer is made from an absorbable material, these effects are temporarily, which permits a control of the healing process after implantation.

If the film layer were non-transparent, it would be generally easy to find out whether the implant is oriented upwardly or downwardly because the basic structure (e.g. a surgical mesh) on top of the film layer would look much different from the film layer on top of the basic structure. In many applications, however, the film layer is transparent so that a corresponding difference in appearance would be small. In the invention, the marker indicates the orientation of the outer face of the film layer (and also indicates the center area of the basic structure) so that the handling of the surgical implant is largely facilitated. If the marker has anti-adhesive properties as well, it will not deteriorate the anti-adhesive effect of the film layer although it is attached to the outer face thereof.

The surgical implant according to the invention is particularly suitable for an intraperitoneal placement. In this case, the basic structure faces the abdominal wall or the peritoneum (parietal side), whereas the film layer is at the visceral side and faces, e.g., the intestine, where it is important to prevent adhesions. Because of the marker, it is relatively easy to place the implant in the correct upside/downside orientation so that the film layer indeed faces, e.g., the intestine. The marker also indicates the center area of the basic structure, which further facilitates the handling of the implant.

In an example for a process of intraperitoneally placing the surgical implant according to the invention in a patient's body, the surgical implant is introduced into the body via a trocar sleeve and is deployed, thereby using the marker to ensure that the second face of the basic structure faces the peritoneum. Then the surgical implant can be fixed on the peritoneum, e.g. by sutures and/or clips. The implant can be used in open surgery as well.

Optionally, before the implant is fixed on the peritoneum, the implant is stuck on the peritoneum (without using any glue) so that it clings to the peritoneum via the second face of the basic structure. This allows for some repositioning before the implant is finally fixed and generally facilitates the procedure. The clinging effect depends on the properties of the face of the implant where the second face of the basic structure is located, and the clinging effect is considerably improved when the film layer is deformed into at least part of the pores of the basic structure so that it forms, in those pores, film regions close to the level of the second face of the basic structure (see below).

The surgical implant according to the invention can be applied, e.g., for repair of a tissue or muscle wall defect, such as a ventral hernia or an inguinal hernia, but also as a hernia mesh in general, as a patch for the dura mater, as a reinforcement patch for staple lines or suture lines in general surgery, as a vessel patch, as a heart patch, or as a reinforced adhesion barrier film.

The marker is attached to the outer face of the film layer, i.e. to that face of the film layer which faces away from the basic structure. This feature provides several advantages: The basic structure is free from marker material, which might impede the in-growth of bodily tissue into the basic structure. The connection between the basic structure and the film layer is not disturbed by the marker, neither mechanically nor with respect to an even geometry. The difference in appearance with respect to upside/downside orientation is more significant because the marker is viewed either directly or through the basic structure plus the film layer.

Another advantage of the surgical implant according to the invention is an easy and efficient way of manufacturing, see below.

In addition to indicating an upside/downside orientation and to indicating a center area of the basic structure, the marker may be adapted to indicate a direction. For example, this direction may be determined by a longitudinal axis of the implant.

In advantageous embodiments of the invention, the marker lacks mirror symmetry about a mirror axis lying in a plane defined by the outer face of the film layer. This feature, combined with a suggestive appearance of the marker, permits an easy determination whether the outer face of the film layer is oriented towards the user or a desired object, respectively, or away therefrom.

For example, the marker may comprise a string of characters, a logo, a direction-indicating line, a center-indicating marking, or a combination of such elements. The marker, e.g., can be constituted by the word “visceral”. In this case, the arrangement of the word can indicate the center of the basic structure (e.g., at the letters “ce”) and a desired direction, and the upside/downside orientation is evident from whether the word appears in mirror-writing or whether not. Such a marker permits an easy recognition of the center area of the basic structure, of the correct upside/downside orientation, and of the correct directional alignment of the implant. Thus, the marker is a considerable aid during a surgical operation.

If the film layer is transparent, the marker may be adapted to appear at different contrasts and/or intensities when viewed either from the side of the outer face of the film layer or from the opposite side. This effect improves the power of distinction with respect to the correct upside/downside orientation.

There are many possibilities for the design, the arrangement and the other features of the marker. For example, the marker may be positioned in a center area of the basic structure, it may have a prominent element in a center area of the basic structure, it may indicate a symmetry axis of the basic structure, and/or it may indicate a pre-determined direction depending on mechanical properties of the basic structure (in particular, if the basic structure is non-isotropic, e.g. because of a warp pattern). Moreover, the marker may be continuous (e.g., essentially the word “visceral” in handwriting) or non-continuous (e.g., the word “visceral” in printed letters). The marker may be palpable, which additionally aids in finding the correct upside/downside orientation. Rounded edge zones of the marker are advantageous to avoid any injury. A lusterless surface of the marker generally avoids unpleasant light reflexes. The marker may also have a textured surface. Whereas in advantageous embodiments the marker is fully laminated to the film layer, a partial lamination to the film layer is conceivable as well. The marker may be thicker than the film layer, even much thicker than the film layer, e.g. by a factor of at least 5 or by a factor of at least 10. If the edge angle of the marker to the film layer is less than 90 degrees (e.g. less than 85 degrees, less than 75 degrees or less than 60 degrees), the marker may relatively smoothly emerge from the film layer. The marker may be generally flat, at least partially. If the film layer is provided with pores, the marker may enter such pores so that it can be directly connected to the basic structure, through the pores, and stay connected after absorption of the film layer. The marker may at least partially follow a topography determined by the film layer.

Concerning the material of the marker, the marker may contain a polymer, e.g. derived from a polymer film. Preferably, the marker comprises absorbable film material. If the marker has a melting temperature which is lower than the melting temperature of the film layer, the marker can be attached to the film layer by a kind of hot-melt process which does not deteriorate the film layer. In advantageous embodiments, the marker has anti-adhesive properties with respect to biological structures so that the masking of an anti-adhesive effect of the outer face of the film layer in the area of the marker is compensated. The marker may be colored to increase its visibility, e.g. colored with a dye which de-colors in mammalian tissue in less than 30 days.

In an embodiment, the marker is made from a poly-p-dioxanone film dyed with a violet dye (e.g., “D&C Violet No. 2”) and having a thickness of approximately 150 μm. To this end, the marker may be cut from an extruded film sheet using common cutting techniques, such as by using a laser, a knife, a cutting die, and/or ultrasound. The marker may be made from one piece of material, which facilitates positioning and orienting the marker on the film layer. In different embodiments (as already mentioned), the marker may be made from multiple pieces and, e.g., kiss-cut on a backing sheet for ease of placement for lamination to the film layer.

In other embodiments, printing or spraying techniques are used to apply the marker to the outer face of the film layer. This can be performed before or after the absorbable film layer is attached to the basic structure. For example, a coloring agent may be prepared by dissolving a dye and a polymer in a suitable solvent, and then the marker is sprayed onto the outer face of the film layer, e.g. by using an air-brush technique or an ink-jet printer. After evaporation of the solvent, the marker is firmly connected to the film layer. The marker prepared in this way may have further properties like being absorbable, having anti-adhesive properties with respect to biological structures, being colored, and/or being colored and de-coloring in mammalian tissue in less than 30 days.

As already mentioned, the film layer generally comprises barrier and anti-adhesive properties so that it can prevent ingrowth of bodily tissue from the visceral side and prevents adhesions to, e.g., the intestine. The film layer may be non-porous, but the barrier and anti-adhesive properties do not necessarily exclude the presence of pores. If the film layer is provided with pores, the marker may enter such pores (e.g. during manufacture at a higher temperature) so that it can be directly connected to the basic structure, through the pores, and stay connected to the basic structure after absorption of the film layer. The film layer may be generally flat, preferably following the configuration determined by the basic structure, or it may be deformed into at least part of the pores of the basic structure. Whereas the film layer may be continuous, e.g. completely covering the first face of the basic structure, alternatively it may be constituted of a plurality of individual film pieces spaced to each other (or touching each other, e.g. in a corner). In the latter case, the marker may be attached to one or to more than one of these film layer pieces.

In embodiments, the film layer is deformed into at least part of the pores of the basic structure where it forms, in a respective pore, a film region close to the level of the second face of the basic structure. Thus, starting from the first face of the basic structure, the film layer enters at least part of the pores and then extends, in the inner area of a pore considered, e.g. roughly at the level of the second face of the basic structure or close thereto, to form a film region inside that pore. In this way, the second face may get largely smooth, because the structure determined by the second face of the basic structure may be generally leveled by the film regions formed by the film layer inside the pores. The effect of general smoothness at the second face has the advantage that the implant can slightly adhere at the peritoneum when the implant is intraperitoneally placed, which significantly facilitates the surgical procedure. In the area where the film layer is covered by the marker, the combination of marker and film layer may be deformed into pores as well.

The basic structure can be designed in multiple ways, as generally known in the art. For example, the basic structure may comprise a surgical mesh (e.g., a warp-knitted mesh), a mesh-like sheet, a spacer fabric, a perforated film, a perforated woven, or a perforated non-woven, or it may be designed as a mesh pouch (e.g. as a surgical mesh wherein part of the mesh is folded to form a pocket). An essential feature of the basic structure is the presence of pores which extend across the thickness of the basic structure. The pores may have a size, e.g., in the range of from 1 mm to 9 mm. Herein, the size of a given pore is defined as the greatest (free) width of that pore. It is also possible that the pores of a given basic structure have different sizes and/or different shapes. The basic structure may be at least partially translucent. Moreover, the basic structure may have anisotropic stretching properties, as already mentioned further above in the context of a direction indicated by the marker. A surgical mesh may be made from monofilaments, multifilaments and/or threads having different diameters/sizes.

In advantageous embodiments of the invention, the basic structure is long-term stable. This can be achieved by non-absorbable materials, which generally are well known in the art. Examples of non-absorbable materials are polyalkenes, polypropylene, polyethylene, fluorinated polyolefins, polytetrafluoroethylene, PTFE, ePTFE, cPTFE, polyvinylidene fluoride, blends of polyvinylidene fluoride and copolymers of vinylidene fluoride and hexafluoropropene, polyamides, polyimides, polyurethanes, polyisoprenes, polystyrenes, polysilicones, polycarbonates, polyarylether ketones, polymethacrylic acid esters, polyacrylic acid esters, aliphatic polyesters, aromatic polyesters, and mixtures of such substances as well as copolymers of polymerizable substances of that list.

As used herein, the terminology long-term stable means a non-absorbable polymer or a very slowly absorbable polymer that still possesses at least 50 percent of its original tearing strength 60 days after implantation.

The basic structure may also comprise absorbable material, either exclusively or in addition to non-absorbable material, e.g. synthetic bioabsorbable polymer materials, polyhydroxy acids, polylactides, polyglycolides, copolymers of glycolide and lactide, copolymers of glycolide and lactide in the ratio 90:10, copolymers of glycolide and lactide in the ratio 5:95, copolymers of lactide and trimethylene carbonate, copolymers of glycolide, lactide and trimethylene carbonate, polyhydroxybutyrates, polyhydroxyvaleriates, polycaprolactones, copolymers of glycolide and ε-caprolactone, polydioxanones, poly-p-dioxanone, synthetic and natural oligo- and polyamino acids, polyphosphazenes, polyanhydrides, polyorthoesters, polyphosphates, polyphosphonates, polyalcohols, polysaccharides, polyethers, collagen, gelatin, bioabsorbable gel films crosslinked with omega 3 fatty acids, oxygenized regenerated cellulose, or mixtures of such substances.

The film layer may be designed as a polymeric barrier film comprising an absorbable material, e.g. copolymers of glycolide and s-caprolactone, collagens, gelatine, hyaluronic acid, polyvinyl pyrrolidone, polyvinyl alcohol, fatty acids, polyhydroxy acids, polyether esters, polydioxanones, or mixtures or copolymers of polymerizable substances thereof. Generally, a non-absorbable material of the film layer (or a mixture of absorbable and non-absorbable materials) is conceivable as well.

The marker may comprise, e.g., poly-p-dioxanone, preferably dyed poly-p-dioxanone, e.g. poly-p-dioxanone dyed with a violet dye (e.g. “D&C Violet No. 2”).

In an advantageous embodiment, the surgical implant comprises a basic structure designed as a macro-porous surgical mesh (about 1 mm to 9 mm maximum extension within each pore) knitted from polypropylene (PROLENE®, Ethicon; non-absorbable) and poly-p-dioxanone (PDS®, Ethicon; absorbable) fibers. An absorbable film made of a copolymer of glycolide and ε-caprolactone (MONOCRYL® (polyglecaprone 25), Ethicon) is laminated to the first face of the basic structure, wherein it extends into the pores and serves as a barrier layer. Additionally, a marker cut from a dyed poly-p-dioxanone film is fixed to the barrier layer, on its outer face, opposite to the surgical mesh. The implant is generally flat and has a “mesh side” and a “film side”.

This surgical implant is a partially absorbable, flexible composite mesh prosthesis intended for the repair of ventral or incisional hernias and other fascial defects, including inguinal hernias. The implant can be placed in an IPOM (intraperitoneal onlay mesh) technique. After intra-peritoneal implantation, the implant is in a permanent tissue contact. It comes into contact with the peritoneum on the parietal side (surgical mesh side) and with intra-abdominal organs on the visceral side (film/barrier layer side). In summary, the structural elements of the implant have the following functions: (1) The non-absorbable polypropylene mesh component of the basic structure is used to reinforce or bridge defects to provide extended support during and following wound healing. (2) The absorbable barrier layer of MONOCRYL® is intended to physically separate the basic structure from underlying tissue and organ surfaces (as bowel/omentum) during the critical wound healing period until the basic structure mesh is covered by a neoperitoneum, thereby reducing the extent and severity of unintended tissue attachment to the permanent material of the basic structure. (3) During laparoscopic placement, the textured pattern formed at the second face of the basic structure by the mesh structure and by the regions of the film layer deformed into the mesh pores provides good clinging properties so that the handling of the implant is much improved. And the marker indicates the correct upside/downside orientation and the center area of the implant in an easily recognizable manner.

Moreover, the surgical implant according to the invention may comprise at least one active ingredient and/or at least one contrast agent, e.g. incorporated in, applied to or adsorbed to the basic structure and/or provided at a layer of the implant, e.g. incorporated in or adsorbed to the film layer, and/or in encapsulated form.

Examples for active ingredients are biologically active or therapeutic ingredients which can optionally be released locally after the implantation. Substances which are suitable as active or therapeutic agents may be naturally occurring or synthetic, and include but are not limited to, for example, antibiotics, antimicrobials, antibacterials, antiseptics, chemotherapeutics, cytostatics, metastasis inhibitors, antidiabetics, antimycotics, gynecological agents, urological agents, anti-allergic agents, sexual hormones, sexual hormone inhibitors, haemostyptics, hormones, peptide-hormones, antidepressants, vitamins such as Vitamin C, antihistamines, naked DNA, plasmid DNA, cationic DNA complexes, RNA, cell constituents, vaccines, cells occurring naturally in the body or genetically modified cells. The active or therapeutic agent may be present in various forms including in an encapsulated form or in an adsorbed form. With such active agents, the patient outcome may be improved or a therapeutic effect may be provided (e.g., better wound healing, or inflammation inhibition or reduction).

One preferred class of active agents are antibiotics that include such agents as gentamicin or ZEVTERA™ (ceftobiprole medocaril) brand antibiotic (available from Basilea Pharmaceutica Ltd., Basel, Switzerland). Other active agents that may be used are highly effective broad-band antimicrobials against different bacteria and yeast (even in the presence of bodily liquids) such as octenidine, octenidine dihydrochloride (available as active ingredient in Octenisept® disinfectant from Schülke & Mayer, Norderstedt, Germany), polyhexamethylene biguanide (PHMB) (available as active ingredient in Lavasept® from Braun, Switzerland), triclosan, copper (Cu), silver (Ag), nanosilver, gold (Au), selenium (Se), gallium (Ga), taurolidine, N-chlorotaurine, alcohol-based antiseptics such as Listerine® mouthwash, N-a-lauryl-L-arginine ethyl ester (LAE), myristamidopropyl dimethylamine (MAPD, available as an active ingredient in SCHERCODINE™ M), oleamidopropyl dimethylamine (OAPD, available as an active ingredient in SCHERCODINE™ O), and stearamidopropyl dimethylamine (SAPD, available as an active ingredient in SCHERCODINE™ S), fatty acid monoesters, taurolidine, and PHMB.

Another class of active agents are local anesthetics that include such agents as: Ambucaine, Benzocaine, Butacaine, Procaine/Benzocaine, Chloroprocaine, Cocaine, Cyclomethycaine, Dimethocaine/Larocaine, Etidocaine, Hydroxyprocaine, Hexylcaine, Isobucaine, Paraethoxycaine, Piperocaine, Procainamide, Propoxycaine, Procaine/Novocaine, Proparacaine, Tetracaine/Amethocaine, Lidocaine, Articaine, Bupivacaine, Dibucaine, Cinchocaine/Dibucaine, Etidocaine, Levobupivacaine, Lidocaine/Lignocaine, Mepivacaine, Metabutoxycaine, Piridocaine, Prilocaine, Propoxycaine, Pyrrocaine, Ropivacaine, Tetracaine, Trimecaine, Tolycaine, combinations thereof, e.g., Lidocaine/prilocaine (EMLA) or naturally derived local anesthetics including Saxitoxin, Tetrodotoxin, Menthol, Eugenol and prodrugs or derivatives thereof.

Moreover, a contrast agent may be included in or on the surgical implant according to the invention. Such a contrast agent may be a gas or gas-creating substance for ultrasound contrast or for MRI contrast, such as metal complexes like GdDTPA or superparamagnetic nanoparticles (Resovistm or Endoremm) as taught in EP 1 324 783 B1, which is incorporated by reference. X-Ray visible substances might be included as shown in EP 1 251 794 B1 (incorporated by reference), including pure zirconium dioxide, stabilized zirconium dioxide, zirconium nitride, zirconium carbide, tantalum, tantalum pentoxide, barium sulphate, silver, silver iodide, gold, platinum, palladium, iridium, copper, ferric oxides, not very magnetic implant steels, non-magnetic implant steels, titanium, alkali iodides, iodated aromatics, iodated aliphatics, iodated oligomers, iodated polymers, alloys of substances thereof capable of being alloyed.

In a process of manufacturing the surgical implant according to the invention, a flexible, areal basic structure having a first face and a second face is provided, with pores extending from the first face to the second face. Moreover, an absorbable film layer is provided, which may serve as a barrier layer. The film layer is placed onto the first face of the basic structure, an outer face of the film layer facing away from the basic structure. By applying heat and pressure, a material provided at the basic structure and/or the film layer is softened and attaches the film layer to the basic structure. If the film layer is to enter the pores of the basic structure to form film regions in the respective pores (see above), a relatively hard support facing the basic structure and a relatively soft pad facing the film layer can be used for applying the pressure. Moreover, an absorbable marker, which is adapted to indicate an upside/downside orientation of the outer face of the film layer and to indicate a center area of the basic structure, is placed on the outer face of the film layer. By applying heat and pressure, a material provided at the film layer and/or the marker is softened and attaches the marker to the film layer. Afterwards, optionally not before the end of a preselected period of time, the temperature and pressure can be decreased.

To improve the attachment between the film layer and the basic structure, a bonding material can be used, which has a melting temperature lower than the melting temperature of at least part of the material of the basic structure and lower than the melting temperature of at least part of the material of the film layer. Such a bonding material melts or gets soft during the application of heat and pressure, thus acting as a kind of melt glue, whereas the basic structure and the film layer are still able to keep their desired shapes.

The bonding material may be included in the basic structure provided, e.g. in the form of filaments comprising poly-p-dioxanone. Alternatively (or additionally), the bonding material may be included in the film layer provided, e.g. as a sub-layer comprising poly-p-dioxanone and laminated to a sublayer comprising barrier material having a higher melting point than poly-p-dioxanone.

When the above-referenced advantageous embodiment of the surgical implant is manufactured, in which the basic structure is knitted from polypropylene and from poly-p-dioxanone fibers, the poly-p-dioxanone fibers serve as the bonding material. The basic structure can be knitted in a way that it is still a stable polypropylene mesh after the poly-p-dioxanone component has lost its shape and structural function during the manufacturing process (and after it has been absorbed after implantation).

Similarly, the marker may comprise poly-p-dioxanone, and it may be attached to the film layer after the film layer has been attached to the basic structure, by applying heat and pressure for a period of time short enough in order to avoid deterioration of the marker, the film layer and the basic structure. This is a two-step process in which heat and pressure are applied twice, (1) to connect the film layer to the basic structure and (2) to attach the marker to the outer face of the film layer. If there is a cool-down period after these steps, even a short one, the attachment between the basic structure and the film layer will not be significantly damaged, in spite of the use of poly-p-dioxanone in the combination of basic structure and film layer and in the marker. Thus, the implant can be manufactured in a convenient and reliable way.

In the following, the invention is further described by means of embodiments. The drawings show in

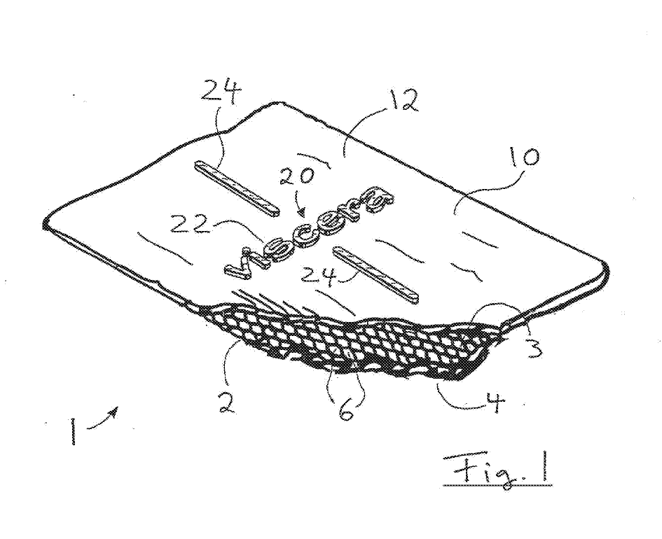

FIG. 1 a three-dimensional view of an embodiment of a surgical implant according to the invention, which is partially cut away, including a first embodiment of a marker,

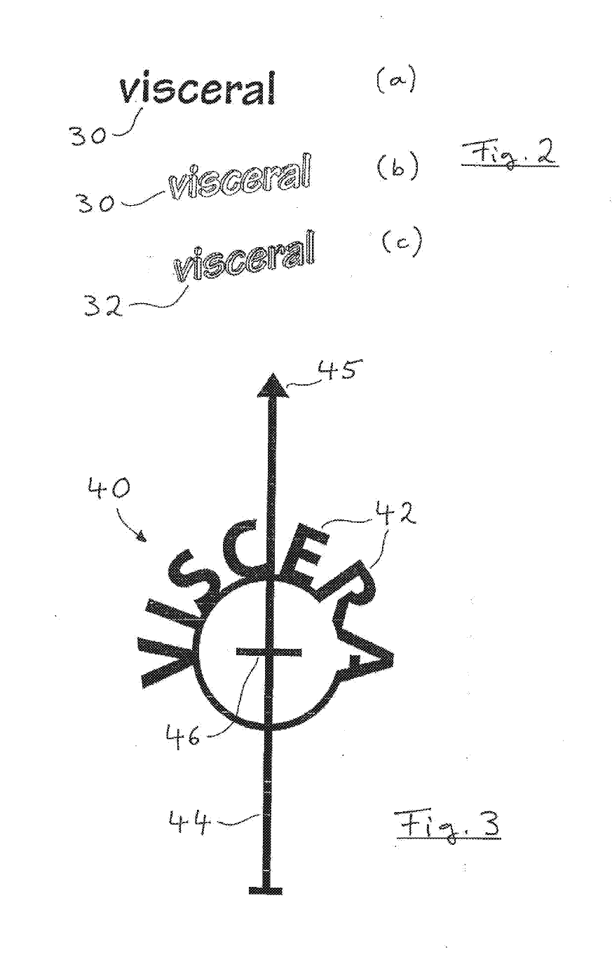

FIG. 2 in part (a) a second embodiment of a marker in plane view, in part (b) the second embodiment of a marker in three-dimensional view, and in part (c) a third embodiment of a marker in three-dimensional view, and

FIG. 3 a fourth embodiment of a marker in plane view.

FIG. 1 illustrates an embodiment of a surgical implant, designated by 1, in a three-dimensional view. The surgical implant 1 comprises a basic structure 2 having a first face 3 and a second face 4. In the view according to FIG. 1, the first face 3 points upwardly and the second face 4 points downwardly. The basic structure 2 includes pores 6 extending from the first face 3 to the second face 4. In the embodiment, the basic structure 2 is designed as a surgical mesh warp-knitted from polypropylene monofilament fibers (non-absorbable) and poly-p-dioxanone (PDS®; Ethicon) monofilament fibers (absorbable).

A film layer 10 is placed at the first face 3 of the basic structure 2 and attached to the basic structure 2. An outer face 12 of the film layer 10 faces away from the basic structure 2. In the embodiment, the film layer 10 is approximately 10 μm thick, transparent and made of MONOCRYL®, an absorbable copolymer of glycolide and s-caprolactone (polyglecaprone 25 suture polymer or MONOCRYL®, Ethicon). The film layer 10 serves as a barrier layer and has anti-adhesive properties. In FIG. 1, the film layer 10 is removed in one corner of the surgical implant 1 to exhibit the basic structure 2.

An absorbable marker 20 is attached to the outer face 12 of the film layer 10. In the embodiment, the marker 20 is made from laser-cut film material (thickness about 100 μm) of PDS® (absorbable) dyed with the biocompatible violet dye “D&C Violet No. 2”.

The marker 20 is a first embodiment of a marker. It comprises a string 22 of characters, forming the word “viscera”, and an interrupted line 24. When the surgical implant 1 is oriented as shown in FIG. 1, the marker 20 is at its upper side, and “viscera” can be easily read. However, if the surgical implant 1 is oriented upside down, the word “viscera” will appear in mirror-writing, thus clearly indicating the different upside/downside orientation. The interrupted line 24 may serve as an indicator of a direction, e.g. for an axis of the surgical implant 1 where the elastic properties behave in a particular way or simply as an indicator for the long side of the rectangular shape of the surgical implant 1. Moreover, the gap in the interrupted line 24 as well as the letters “ce” indicate the center area of the surgical implant 1.

FIG. 2(a) shows a second embodiment of a marker, designated by 30. The marker 30 consists of the word “visceral” only, which is able to indicate the correct upside/downside orientation of the surgical implant (mirror-writing or not), its center area (letters “ce”) and an orientation (direction of writing). The three-dimensional view of the marker 30 in FIG. 2(b) illustrates that the letters of “visceral” have relatively sharp edges. A similar marker 32, shown as a third embodiment in FIG. 2(c), is made of letters comprising roundish edges, which may improve palpability.

A fourth embodiment of a marker, designated by 40, is displayed in FIG. 3 in plane view. The marker 40 is more elaborate and, in contrast to the markers 20, 30 and 32, made in one piece. It comprises a string 42 of characters to indicate the upside/downside orientation, a line 44 with an arrow 45 to indicate a direction, and a particular center marking 46.

Generally, markers having the desired properties can be designed in multiple ways.

The following example describes the manufacture of an embodiment of the surgical implant according to the invention.

### EXAMPLE 1

A composite surgical mesh, comprising non-absorbable monofilament polypropylene and dyed absorbable monofilament PDS® fibers and serving as a basic structure, was heat-laminated with a 10-μm MONOCRYL® film (serving as an adhesion barrier layer) in a lamination press, wherein the PDS® fibers acted as a melt glue. After cooling down under pressure, a 150-μm PDS® film dyed with “D&C Violet No. 2” (serving as a marker) was placed on top of the MONOCRYL® film, covered with a rough baking paper and laminated (for some seconds, at about 110° C., and at a low pressure of 1-5 bar), wherein the pressing period was shorter than in the first lamination step. The marker was firmly attached to the underlying MONOCRYL® film of the surgical implant manufactured in this way. Under the microscope, the surface of the marker showed a micro-roughness (due to the rough baking paper) with tiny lines in multiple directions having a width of less than 20 μm.

In an intraperitoneal pig handling study, this surgical implant was easily passed through a trocar sleeve and placed at the abdominal wall. It showed a clinging effect, and the absorbable marker indicated the correct “north-south” direction and additionally the visceral side. The visibility of the marker was intense when the surgical implant was correctly positioned. A wrong upside/downside orientation strongly reduced the intense visibility impression of the marker, thus providing an additional orientation means.

### EXAMPLE 2

Samples of surgical implants were prepared according to Example 1, each one having a rectangular size of 3 cm×5 cm with slightly rounded edges and having one additional round dyed PDS® disk film (thickness about 150 μm) centrally laminated on the outer face of the MONOCRYL® film.

A rabbit peritoneal defect model was used, as described elsewhere (U.S. Pat. No. 8,629,314 B). Adhesions where evaluated after 2 weeks, see Table 1.

When a respective implant was correctly placed, with the mesh side to the abdominal wall and the film side to the viscera, almost no adhesions occurred. Only one implantation site (out of eight) showed minor grade 1 adhesion (12.5% incidence), the remaining test samples were free of adhesion. However, when the implant was wrongly positioned with the mesh side facing to the viscera, in 87.5% of the cases adhesion occurred, generally more severe with grades from 1 to 4.

Surprisingly, the inventive surgical implants with the film side and the marker facing to the viscera exhibited a good adhesion reduction. After explantation, the marker polymer was still present, but its color had completely leached of, making the marker almost invisible.

TABLE 1
In-vivo performance (rabbit, sidewall model)

| Treatment Groups | | |
|---|---|---|
| (n = 8) | Adhesion Incidence | Adhesion Extent |
| Sham Control | 8/8 (100%) | Grade 1 (2/8) |
| | | Grade 2 (4/8) |
| | | Grade 3 (1/8) |
| | | Grade 4 (1/8) |
| Example 2 (film side | 1/8 (12.5%) | Grade 0 (7/8) |
| to viscera) | | Grade 1 (1/8) |
| Example 2 (mesh side | 7/8 (87.5%) | Grade 0 (1/8) |
| to viscera) | | Grade 1 (3/8) |
| | | Grade 2 (2/8) |
| | | Grade 4 (2/8) |

1. A surgical implant, comprising
a flexible, areal basic structure having a first face and a second face and being provided with pores extending from the first face to the second face, and
an absorbable film layer placed at the first face of the basic structure, being attached to the basic structure and having an outer face facing away from the basic structure,
characterized by an absorbable marker attached to the outer face of the film layer, wherein the marker is adapted to indicate an upside/downside orientation of the outer face of the film layer and to indicate a center area of the basic structure.

2. A surgical implant according to claim 1, characterized in that the marker is adapted to indicate a direction.

3. A surgical implant according to claim 1, characterized in that the marker lacks mirror symmetry about a mirror axis lying in a plane defined by the outer face of the film layer.

4. A surgical implant according to any one of claim 1, characterized in that the marker comprises at least one of the following elements: string of characters logo, direction-indicating line, center-indicating marking.

5. A surgical implant according to any one of claim 1, characterized in that the film layer is transparent.

6. A surgical implant according to claim 5, characterized in that the marker is adapted to appear at different contrasts and/or intensities when viewed either from the side of the outer face of the film layer or from the opposite side.

7. A surgical implant according to any one of claim 1, characterized in that the marker comprises at least one of the following features: being positioned in a center area of the basic structure, having a prominent element in a center area of the basic structure, indicating a symmetry axis of the basic structure, indicating a pre-determined direction depending on mechanical properties of the basic structure, being continuous, being non-continuous, being palpable, having rounded edge zones, having a lusterless surface, having a textured surface, being fully laminated to the film layer, being partially laminated to the film layer, being thicker than the film layer, being thicker than the film layer by a factor of at least 5, being thicker than the film layer by a factor of at least 10, having an edge angle to the film layer of less than 90 degrees, having an edge angle to the film layer of less than 75 degrees, having an edge angle to the film layer of less than 60 degrees, at least partially being generally flat, entering pores provided in the film layer at least partially following a topography determined by the film layer.

8. A surgical implant according to any one of claim 1, characterized in that the marker comprises at least one of the following features: containing a polymer, being derived from a polymer film, comprising absorbable film material, having a melting temperature which is lower than the melting temperature of the film layer, having anti-adhesive properties with respect to biological structures, being colored, being colored and de-coloring in mammalian tissue in less than 30 days.

9. A surgical implant according to any one of claim 1, characterized in that the marker is applied to the outer face of the film layer by using a printing or spraying technique.

10. A surgical implant according to any one of claim 1, characterized in that the film layer is deformed into at least part of the pores of the basic structure where it forms, in a respective pore, a film region close to the level of the second face of the basic structure.

11. A surgical implant according to any one of claim 1, characterized in that the film layer comprises one of the features in each one of the following sets of contrasting features: being non-porous, having pores; being generally flat, being deformed into at least part of the pores of the basic structure; being continuous, being made of a plurality of spaced film pieces.

12. A surgical implant according to any one of claim 1, characterized in that the film layer comprises pores, wherein material of the marker penetrates at least part of the pores and is connected to the basic structure.

13. A surgical implant according to any one of claim 1, characterized in that the basic structure comprises at least one of the following features: comprising perforated film material, comprising a surgical mesh, being at least partially translucent, having anisotropic stretching properties.

14. A surgical implant according to any one of claim 1, characterized in that the basic structure comprises a non-absorbable material, preferably at least one of the materials selected from the following list: polyalkenes, polypropylene, polyethylene, fluorinated polyolefins, polytetrafluoroethylene, PTFE, ePTFE, cPTFE, polyvinylidene fluoride, blends of polyvinylidene fluoride and copolymers of vinylidene fluoride and hexafluoropropene, polyamides, polyimides, polyurethanes, polyisoprenes, polystyrenes, polysilicones, polycarbonates, polyarylether ketones, polymethacrylic acid esters, polyacrylic acid esters, aliphatic polyesters, aromatic polyesters, copolymers of polymerizable substances thereof.

15. A surgical implant according to any one of claim 1, characterized in that the basic structure comprises an absorbable material, preferably at least one of the materials selected from the following list: synthetic bioabsorbable polymer materials, polyhydroxy acids, polylactides, polyglycolides, copolymers of glycolide and lactide, copolymers of glycolide and lactide in the ratio 90:10, copolymers of glycolide and lactide in the ratio 5:95, copolymers of lactide and trimethylene carbonate, copolymers of glycolide, lactide and trimethylene carbonate, polyhydroxybutyrates, polyhydroxyvaleriates, polycaprolactones, copolymers of glycolide and ε-caprolactone, polydioxanones, poly-p-dioxanone, synthetic and natural oligo- and polyamino acids, polyphosphazenes, polyanhydrides, polyorthoesters, polyphosphates, polyphosphonates, polyalcohols, polysaccharides, polyethers, collagen, gelatin, bioabsorbable gel films cross-linked with omega 3 fatty acids, oxygenized regenerated cellulose.

16. A surgical implant according to any one of claim 1, characterized in that the film layer comprises at least one of the materials selected from the following list: copolymers of glycolide and ε-caprolactone, collagens, gelatine, hyaluronic acid, polyvinyl pyrrolidone, polyvinyl alcohol, fatty acids, polyhydroxy acids, polyether esters, polydioxanones, copolymers of polymerizable substances thereof.

17. A surgical implant according to any one of claim 1, characterized in that the marker comprises at least one of the materials selected from the following list: poly-p-dioxanone, dyed poly-p-dioxanone, poly-p-dioxanone dyed with a violet dye.

18. A process of manufacturing a surgical implant according to claim 1, characterized by the steps:
providing a flexible, areal basic structure having a first face and a second face and being provided with pores extending from the first face to the second face,
providing an absorbable film layer,
placing the film layer onto the first face of the basic structure, an outer face of the film layer facing away from the basic structure,
applying heat and pressure, thereby softening a material provided at at least one of the basic structure and the film layer and attaching the film layer to the basic structure,
providing an absorbable marker which is adapted to indicate an upside/downside orientation of the outer face of the film layer and to indicate a center area of the basic structure,
placing the marker on the outer face of the film layer,
applying heat and pressure, thereby softening a material provided at at least one of the film layer and the marker and attaching the marker to the film layer.

19. A process according to claim 18, characterized in that at least one of the basic structure and the film layer comprises a bonding material, which has a melting temperature lower than the melting temperature of at least part of the material of the basic structure and lower than the melting temperature of at least part of the material of the film layer wherein the bonding material preferably comprises poly-p-dioxanone.

20. A process according to claim 18, characterized in that the marker comprises poly-p-dioxanone and is attached to the film layer, after the film layer has been attached to the basic structure, by applying heat and pressure for a period of time short enough in order to avoid deterioration of the marker, the film layer and the basic structure.

21. A process of intraperitoneally placing a surgical implant according to any one of claim 1 in a patient's body, comprising the steps:
introducing the surgical implant via a trocar sleeve into the body and deploying the surgical implant, thereby using the marker to ensure that the second face of the basic structure faces the peritoneum,
fixing the implant on the peritoneum.

22. A process according to claim 21, wherein the implant, with the second face of the basic structure, is stuck on the peritoneum before the implant is fixed on the peritoneum.
